SEQUENCE LISTING (2 sequences)

SEQ ID NO: 1 - protein, 4 aa, Artificial Sequence
  Description of Artificial Sequenceamino acid sequence within molecular recognition switch, target for melanoma cell binding
REDV

SEQ ID NO: 2 - protein, 5 aa, Artificial Sequence
  Description of Artificial Sequenceamino acid sequence within molecular recognition switch serving as binding site for mammary tumor cells
FRGDS

(19) United States
(12) Patent Application Publication
(10) Pub. No.: US 2003/0186323 A1
(43) Pub. Date: Oct. 2, 2003

(54) Force-regulated molecular recognition switches

(76) Inventors: Viola Vogel, Seattle, WA (US); Andre Krammer, Seattle, WA (US); Klaus Schulten, Urbana, IL (US); Barry Isralewitz, Urbana, IL (US); Hui Lu, Urbana, IL (US)

Correspondence Address: GREENLEE WINNER AND SULLIVAN P C, 5370 MANHATTAN CIRCLE, SUITE 201, BOULDER, CO, 80303, US

(21) Appl. No.: 10/229,820

(22) Filed: Aug. 26, 2002

Related U.S. Application Data
(63) Continuation of application No. 09/335,118, filed on Jun. 17, 1999, now abandoned.
(60) Provisional application No. 60/089,665, filed on Jun. 17, 1998.

(51) Int. Cl. G01N 33/53; C12M 1/34; G06F 19/00; G01N 33/48; G01N 33/50

(52) U.S. Cl. 435/7.1; 435/287.2; 702/19

(57) ABSTRACT

The present disclosure provides force-regulated molecular switches and methods for controlling binding and release of a ligand (cell, protein or other polymer, or small molecule) to the switch-containing device by the application, release or modulation of force (physical tension or an electrical or magnetic field as specifically exemplified herein). The FRMR switch technology can be applied to vectorial pumps, molecule-specific sponges, calorimetric cell motility assays, electronically addressable biorecognition arrays, cell sorting devices, tissue engineering scaffolds, calorimetric affinity assays, diagnostics and therapeutics.

### CROSS REFERENCE TO RELATED APPLICATIONS

This application is a continuation of U.S. application Ser. No. 09/335,118 filed Jun. 17, 1999, which claims benefit of U.S. Provisional Application No. 60/089,665, filed Jun. 17, 1998, both of which prior applications are incorporated by referenced herein to the extent consistent with the present disclosure.

### ACKNOWLEDGEMENT OF FEDERAL RESEARCH SUPPORT

This invention was made, at least in part, with funding from the National Institutes of Health and the National Science Foundation. Accordingly, the United States Government has certain rights in the invention.

### BACKGROUND OF THE INVENTION

The field of the present invention is the area of nanoscale devices, especially as related to force-regulated molecular recognition switches based on protein scaffolds.

### SUMMARY OF THE INVENTION

The present invention provides methods for making force-regulated molecular recognition (FRMR) switches. A nanoscale switch of the present invention is a biological analog of a transistor, the difference being that the switch can be addressed by mechanical, magnetic or electromagnetic force as well as electricity, and that the regulated signal can be a biorecognition event rather than current. Whereas chemical signaling has been used in the past to regulate biorecognition, we outline methods in which force (mechanical, electrical, magnetic or electromagnetic) applied to a device containing FRMR switches is utilized to regulate biorecognition. All the embodiments of the invention described below have in common that the FRMR modules are either recombinantly expressed or made by solid phase peptide synthesis. The FRMR modules are linked covalently or by high affinity binding to other molecular units or devices in a way that force can be applied to induce at least a partial unfolding of the module's secondary or tertiary structure. Molecular units functionalized by FRMR switch modules include elastic fibers, elastic membranes, elastic scaffold, swellable hydrogels, polymeric matrices or polymeric coatings (e.g., thin films) on elastic-deformable surfaces, piezoelectric devices, and micro- or nanofabricated devices containing movable parts and micro- or nanofabricated devices in which electric or magnetic fields can be applied across FRMR switches. The FRMR switch modules contain one or more loops functionalized with a molecular recognition site, for example, peptide sequences made of natural or non-natural amino acids. In cases where a rapid regeneration of the FRMR is desired, these signaling sequences are preferentially located in loops that connect a helices or β-strands or β-sheets or β-barrels that are pulled out in early stages of the forced unfolding path of the FRMR module. The switches can be designed to be reversible. The recognition element and the protein scaffold can be engineered and further functionalized, and fusion proteins can be generated that contain at least one of these FRMR switches. Areas of principal use of force-regulated molecular recognition switches include applications that take advantage of recombinantly expressed proteins as force-regulated recognition switches; medical applications where FRMR switches are used as therapeutics or in diagnostics; sensors and arrays, medical implants, drug delivery devices and other fields where surfaces are functionalized with molecules that contain at least one FRMR switch in order to regulate binding strength by applying tension, synthetic or biological materials that contain FRMR switches in their interior such that they release or bind molecules after a tension is applied on a local or global scale; applications where FRMR modules are functionalized with fluorophor s, charged particles, magnetic beads or other nanoparticles that are either used to apply an external force upon the FRMR switch, and/or allow the modules to be used as reporters to translate a forced-unfolding event into an optical, electric, magnetic, or other signal. In general, molecular binding to the FRMR switches as described above also includes binding to cell surface molecules as well as to transmembrane proteins. The FRMR switches can be incorporated in polymeric films or matrices, which can further comprise networks, fibers, fibrils and membranes to which the disruptive force can be applied.

Furthermore, more complex FRMR switches can be designed. More complex biological recognition events often require that various recognition sites are exposed in a spatially well defined geometry. Cell adhesion to fibronectin, for example, is further enhanced if the tripeptide sequence RGD on module FnIII₁₀ is simultaneously exposed with the synergy site located on module FnIII₉. Accordingly, the FRMR switch can also contain multiple domains such that a biorecognition event is triggered through simultaneous exposure of at least two signal sequences in a spatially well defined geometry. Through a forced-unfolding event of at least one module within the FRMR, the spatial distances of recognition sites is altered leading to a decreased binding affinity, as shown in FIG. 8.

A device for determining relative binding affinity for ligands and binding partners is provided wherein said device comprises a first surface on which is deposited a thin film comprising a multiplicity of FRMR switches and a second surface on which is immobilized an array of ligands, such as test molecules, wherein each FRMR switch contains a recognition site and an integrated donor/acceptor pair, such that when the first surface having the thin film is first brought into contact with the second surface having the array of test molecules, an adhesive contact between the first and second surfaces results, followed by rapid separation of said surfaces, and separation results in a color change of fluorescence emission spectrum of said donor/acceptor pair, whereby areas of high affinity binding between a ligand on the array and the binding partner of the FRMR switch are identified.

### BRIEF DESCRIPTION OF THE DRAWINGS

FIG. 1 diagrammatically illustrates the tertiary structure of the type III₁₀ repeat of human plasma fibronectin (FnIII₁₀). β-sheets are highlighted by different hatchings. RGD (single letter code) motif is shown in stick-ball representation at the apex of loop FG.

FIGS. 2A-2C show the force-regulated molecular recognition mechanism. The β-strand G (vertically hatched) is pulled out of the scaffold. FIG. 2A shows the structure without force applied. FIG. 2B shows tension applied, with the loop beginning to be deformed, and FIG. 2C shows the loop unfolded after a critical force threshold is overcome.

FIGS. 3A-3F show progressive views of a vectorial molecule-specific pump. FIG. 3A shows the array, with eight FRMR switches, at rest. The curled lines represent folded fibronectin, wherein the RGD at the end of the loop can bind its ligand, integrin, represented by a filled circle. The open rectangles represent electrodes (turned off). FIG. 3B shows diffusion of integrin onto FRMRS 1. In FIG. 3C, voltage is applied across FRMRS 1 to stretch the switch. Electrodes (on) are represented by filled rectangles. Integrin is released from FRMRS 1 and diffuses away from FRMRS 1. The stretched switched is represented by a straight line. In FIG. 3D, integrin diffuses and binds to FRMRS 2. In FIG. 3E, voltage is applied to stretch FRMRS 2. Integrin is released and it diffuses, but it cannot bind to FRMRS 1 or FRMRS 2 in their stretched configurations. FIG. 3F shows binding of integrin to FRMRS 3. Voltage is released from FRMRS 1, which returns to the unstretched loop configuration, which is now capable of binding another integrin molecule.

FIGS. 4A-4B illustrate a stretch-activated scaffold for tissue engineering. A thin film containing covalently linked FRMRSs in the stretched (FIG. 4B) and unstretched (FIG. 4A) modes. In FIG. 4A cells are bound to cell recognition sites (black circles), which function as FRMRSs. When stretch-activated, the cell recognition sites are under tension (black ovals) and undergo a conformational change which prevents cell binding and/or releases cells which had been bound prior to stretch-activation. When the cells are released, they migrate within the stretch-activated scaffold and ultimately can exit the scaffold.

FIGS. 5A-5H diagrammatically illustrate how FRMRSs can be utilized in a calorimetric cell motility assay. As illustrated in FIG. 5A, the FRMRS (black circle) is part of a larger molecule. The FRMRS is functionalized with an energy donor (D) and acceptor (A) pair with a relative distance less than 100 Å. This functionalized FRMRS is then added to a cell culture, for example, growing on a solid support (FIG. 5B). Cells integrate these functionalized FRMRSs into their ECM fibrils, for example, into their fibronectin fibrils (FIG. 5C). Fluorescence resonance energy transfer (FRET) occurs between the D/A pair of the FRMRSs of cells when irradiated with light of wavelength absorbed by the D moiety (FIGS. 5E and 5G). Upon excitation of D by light of an appropriate wavelength, stretch-activation leads to a reduced FRET as the distance between the D/A pair is increased upon stretching (5F and 5H). An increased D/A distance and therefore, a reduced FRET, results in a change of the emission spectrum as outlined in FIG. 5F.

FIGS. 6A-6E schematically illustrate an electronically addressable array of biorecognition sites. A series of FRMRSs, each flanked by a pair of charged beads or segments, are incorporated into a thin film which is deposited on the surface of the electronically addressable array. Application of an electrical field (arrows) across the FRMRS stretch-activates the switch in a localized area (FIG. 6A). This device can now be used in various settings. In one specific example, the FRMRS contains the RGD sequence. Cells are then plated on the surface of the device, with no force exerted on the switches (FIGS. 6B-6C, left). They are exposed in a spatially controlled fashion to drugs, pollutants, or other ligands (generically described herein as biologically active molecules). Spatial control of exposure can be accomplished through the use of solute flow through capillaries (FIG. 6C). On the left, the cells on the surface of the device are then exposed to biologically active molecules in the solute flow. On the right in FIGS. 6B-6C, the cells are added after the solute flow. The cell bed is then exposed to markers (small black balls, FIG. 6D) that test, for example, for cell survival, cell death, cell cycle progression, gene expression, expression of receptor molecules. After analysis of the cell array, cells of interest can be selectively detached from the array by the application of a voltage to the electrodes (FIG. 6E). The potential stretch-activates the FRMRS, thus releasing the cells. Alternatively, the surface of the array can be precoated by drugs, toxins, pollutants or other potential ligands in a spatially controlled manner (FIGS. 6B-6C, right) prior to plating the cells, followed by the procedure essentially as described above.

FIGS. 7A-7D illustrate the details of the FRMRS application to a calorimetric array-based affinity assay. FIG. 7A schematically illustrates the FRMRS, containing acceptor (A), donor (D) and recognition site (R), which is incorporated into a polymeric film. This film is then deposited on top of an array of test molecules (see FIG. 7B, side view). The polymer film is then ripped off the array. The FRMR switches in areas of strong adhesion will be stretch-activated (FIG. 7C). As discussed in FIG. 5, regions within the polymer film that contain stretch-activated FRMR switches give rise to a blue-shifted emission spectrum. Areas where target compounds are bound with high affinity are characterized by color change (cross-hatched areas).

FIG. 8 shows the forced unfolding of an FRMR switch containing two domains, modules FnIII₉ and FnIII₁₀. The distance between the synergy site on FnIII₉ and the RGD-loop on FnIII₁₀ is 30 Å under equilibrium conditions. FIG. 8 illustrates the tertiary structure of this two-switch-containing polypeptide having two ligand binding sites which function as FRMRSs. When the polypeptide is completely folded, there is synergy between the two sites, which are about 30 Å apart. When the tertiary structure of the polypeptide is disrupted by stretch-activation due to applied force to a portion of one of the switches, the two sites are pulled apart (to at least about 50 Å) and at least one of two bound ligands is released, with the result that ligand binding affinity is decreased at both sites. This example is the fibronectin-integrin model.

### DETAILED DESCRIPTION OF THE INVENTION

While major progress has been achieved in the past decades to elucidate how chemical factors regulate biochemical processes, we discovered that force can be utilized to regulate molecular recognition events involving protein modules (Krammer et al. [1999] Proc. Natl. Acad. Sci. USA 96:1351-1356). The understanding of how force can regulate molecular recognition and signaling is still rudimentary due to the fact that high resolution crystallographic structures of biomolecules solely grant access to relaxed equilibrium states. Only two years ago, the first experiments were conducted that allowed measurement of the force necessary to unfold single proteins. This was accomplished by applying a force to their terminal ends using atomic force microscopy and optical tweezers (Rief et al. [1998] Biophys. J 75:3008-3014; Rief et al. [1997] Science 276:1109-1112; Oberhauser et al. [1998] Nature 393:181-185; Kellermayer et al. [1997] Science 276:1112-1116; Tskhovrebova et al. [1997] Nature 387:308-312; Rief et al. [1999] J. Mol. Biol 286:553-561; Carrion-Vasquez et al. [1999] Proc. Natl. Acad. Sci. USA 96:3694-3699; Schemmer and Gaub [1999] Rev. Sci. Instr. 70:1313-1317; Kellermayer et al. [1998] J. Struct. Biol. 122:197-205). These measurements, however, do not provide insight into the unfolding pathway by which the secondary or tertiary structure of proteins unravels if force is applied above a threshold value. Furthermore, no other experimental technique is currently available to visualize how a single force-regulated molecular switch operates on an atomic scale. Here, steered molecular dynamics (SMD) simulations provide fundamentally new insights into force-induced transient conformational states. Using a crystallographic protein structure as the starting point for the simulation, tension is applied to the terminal ends of the molecule through an external harmonic or constant force constraint. Our now well-established SMD simulations of the forced unfolding pathway of proteins have successfully reproduced the experimental finding of a single force peak that has to be overcome to unravel the tertiary structure of β-sandwich modules. This was illustrated using the titin module I27 and fibronectin's type III-10 module as examples (Lu et al. [1998] Biophys. J. 75:662-671; Krammer et al. [1999] supra). Lately, we could also correlate the potential energy barrier along the trajectory of the unfolding pathway, which is of the same order as experimental findings obtained from atomic force measurements. SMD simulations have thus reached a point where new insight can be gained from computational methods about the pathway by which proteins unfold. They are the only available tool to explore in detail how the folding scaffold of a protein behaves when exposed to external forces (Lu and Schulten [1999] Proteins. Structure, Function, Genetics 35:453-463; Krammer et al. [1999] supra; Lu et al. [1998] supra; Izrailev et al. [998] In: Computational Molecular Dynamics: Challenges, Methods, Ideas, Vol. 4 of Lecture Notes in Computational Science and Engineering, Springer-Verlag, Berlin, pp 36-62; Grubmüller et al. [1996]) Science 271:997-999; Izrailev et al. [1997] Biophys. J. 72:1568-1581; Isralewitz et al. [1997] Biophys. J 73:2972-2979; Kosztin et al. [1999] Biophys. J 76:188-197; Stepaniants et al. [1997] J. Mol. Model. 3:473-475).

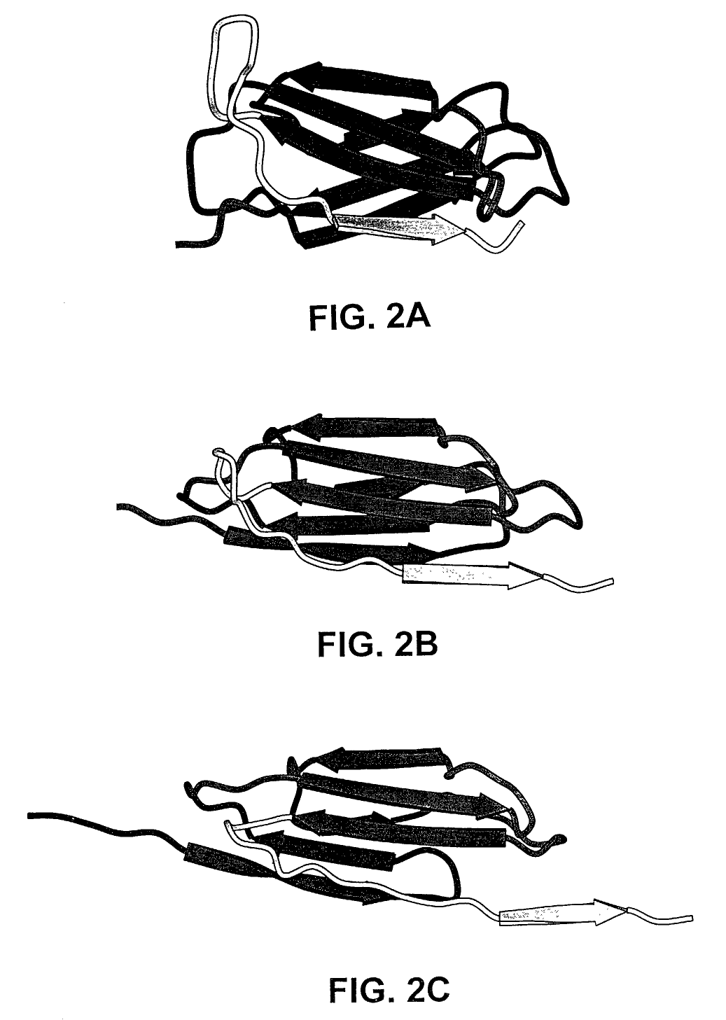

By the use of SMD simulations, as shown in FIGS. 2A-2C, the tenth fibronectin type III (FnIII₁₀) module, which is 94 amino acids long, is stretched from its initially compact and folded structure to a fully elongated configuration at an extension of 310 Å. In the depicted simulation, the N-terminal C_{α} atom (Val1) of the FnIII₁₀ domain is constrained in its motion while the C-terminal C_{α} atom (Thr94) is pulled on with a constant force load. Similar results are obtained in the case of pulling on the N-terminus and holding the C-terminus fixed, as well as simultaneously pulling on both termini. Upon extension of the FnIII₁₀ domain, a single pronounced burst of its structure is observed in our simulations at an extension of about 35 Å. It is known that the force needed to unravel a module scales with the pulling speed (Evans, E. and Ritchie, K. [1997] Biophys. J. 72:1541-1555; Evans and Ritchie [1999] Biophys. J. 76:2439-2447). Our computer simulations require a force of about 1500 pico Newton in order to burst and unfold the module in a computationally feasible time (Krammer et al. [1999] supra). The force required to unravel β-sheet protein motifs has been measured by AFM and optical tweezers studies, revealing forces in the range of 20 to 300 pN for typical pulling velocities (Rief et al. [1998] Biophys. J 75:3008-3014; Rief et al. [1997] Science 276:1109-1112; Oberhauser et al. [1998] Nature 393:181-185; Kellermayer et al. [1997] Science 276:1112-1116; Tskhovrebova et al. [1997] Nature 387:308-312; Rief et al. [1999] J. Mol. Biol. 286:553-561; Carrion-Vasquez et al. [1999] Proc. Natl. Acad. Sci. USA 96:3694-3699; Schemmer and Gaub [1999] Rev. Sci. Instr. 70:1313-1317; Kellermayer et al. [1998] J. Struct. Biol. 122:197-205).

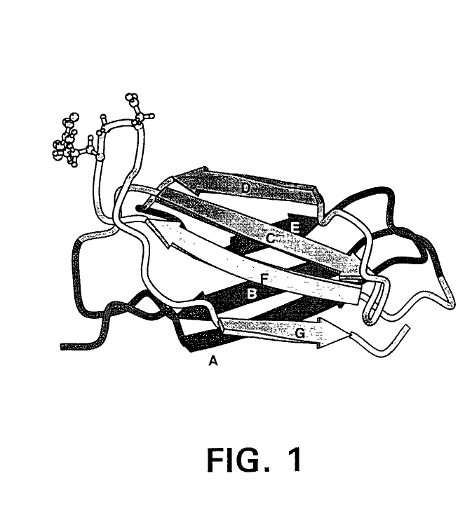

Since the structure of the protein module FnIII₁₀, as shown in FIG. 1, has a scaffold which we discovered is well-suited for the rational design of FRMR switches, we now briefly describe some relevant structural background information. Fibronectin, a glycoprotein of 450-500 kD, is composed of a linear sequence of repeating modules of only three structural motifs. The primary structure of fibronectin is well documented (R. Hynes [1990] Fibronectins, Springer-Verlag, New York). The tertiary structure of FnIII₁₀, which belongs to the type III motif, consists of two antiparallel β-sheets that contain the β-strands ABE and DCFG, respectively. The two β-sheets fold up to form a β-sandwich that is stabilized by intra- and inter-β-strand hydrogen bonds, as well as by hydrophobic interactions among the core residues of FnIII₁₀. FnIII₁₀ displays amino acid sequence homology of at least 87% among various species (human, rat, and bovine). The short peptide of arginine, glycine and aspartic acid, in single letter code RGD, plays a central role in promoting cell adhesion to synthetic and biological surfaces. The RGD is located in the loop connecting the β-strands F and G. The RGD sequence, as well as the type III module of fibronectin, has first been identified in fibronectin, but it is also found in many other proteins. The modules are repeated in multiple tandem copies connected by short linker sequences. Only a single repeat contains the RGD sequence, namely FnIII₁₀. The RGD sequence mediates cell attachment to surfaces by specific binding to transmembrane proteins of the integrin family.

Compelling experimental evidence exists in the literature confirming the notion that FnIII₀ acts as a force-regulated molecular recognition switch, namely the RGD loop is positioned strategically, by connecting the last two terminal P-strands, the length of the RGD loop regulates the affinity of RGD to various members of the integrin family, and finally the specificity by which the RGD binds integrins is reduced if the conformational constraint of the loop is loosened (Carr et al. [1997] Structure [London] 5:949-959). However, before now the FnIII₁₀ module has not been contemplated as a dynamic regulatable unit where the affinity and accessibility to integrins can be regulated by stretching the module.

More detailed experimental observations are outlined below that support our conclusions and thus design criteria derived from SMD simulations.

A common molecular scaffold for the unrelated antibody fragment (OPG2) contains an RYD sequence (Ely et al. [1995] Protein Engineering 8:823-827). OPG2 is a member of the immunoglobulin (Ig) superfamily which has evolved convergent scaffolds with only 20% sequence homology to FnIII₁₀. It is of interest that the RYD sequence in OPG2 is also found in the FG loop connecting the last two β-strands. This illustrates that the FG loop occupies a strategic position.

The RGD motif in FnIII₁₀ is found on a hairpin-like loop that extends about 10 Å away from the outer surface of the molecule. In all cases so far described in literature, the RGD loops have the same general B-turn structure, and RGD is typically found at the apex of a long loop exposed to solvent. Binding assays utilizing RGD peptides coupled to beads via linkers of various sizes revealed that the recognition of the RGD sequence by α_{IIb}β₃ integrins is optimized by a linker length ranging from 10-30 Å (Beer et al. [1992] Blood 79:117-128).

The cyclic conformational restrained synthetic peptides that contain the RGD sequence are partially receptor selective and bind with higher affinity than their linear counterparts (Pierschbacher et al. [1987] J Biol. Chem. 262:17294-17298; Scarborough et al. [1993] J. Biol. Chem. 268:1066-1073; Nowlin et al. [1993] J. Biol. Chem. 268:20352-20359). Integrin binding to other RGD-containing proteins is also reported to be significantly increased when the RGD sequence in the loop was conformationally restricted by a disulfide bond formed between cysteines flanking the RGD sequence (Yamada et al. [1995] J. Biol. Chem. 270:5687-5690).

Finally, it has been shown recently that cells can actively stretch fibronectin fibrils that are part of their extracellular matrix to about four times of their equilibrium length. Since fibronectin is assumed to exist in an extended configuration within the fibrils, a four-time elastic elongation implies that some fibronectin modules unfold under the tension produced by single cells (Ohashi et al. [1999] Proc. Natl. Acad. Sci. USA 96:2153-2158; Hynes, R. O. [1999] Proc. Natl. Acad. Sci. USA 96:2588-2590).

We describe herein how protein scaffolds can be utilized as FRMR switches. We illustrate the principle by using β-sheet modules as scaffolds. This invention, however, includes the use of other tertiary structures like β-barrels, bundles of a-helices, and modules containing both β-strands and α-helices. Key components of the FRMR switch include at least one protein scaffold and at least one ligand binding site, and molecules or devices by which external force is applied to the FRMR modules. The function of the switch is then regulated by the application of force.

For example, one can use a β-sandwich motif where the recognition element is located in a loop connecting two β-strands. Rapid refolding of the FRMR switch can be accomplished if the loop that contains the recognition site is located between β-strands that are pulled out of the scaffold in an early stage of the forced unfolding pathway, while the overall integrity of the remaining module is mostly unperturbed.

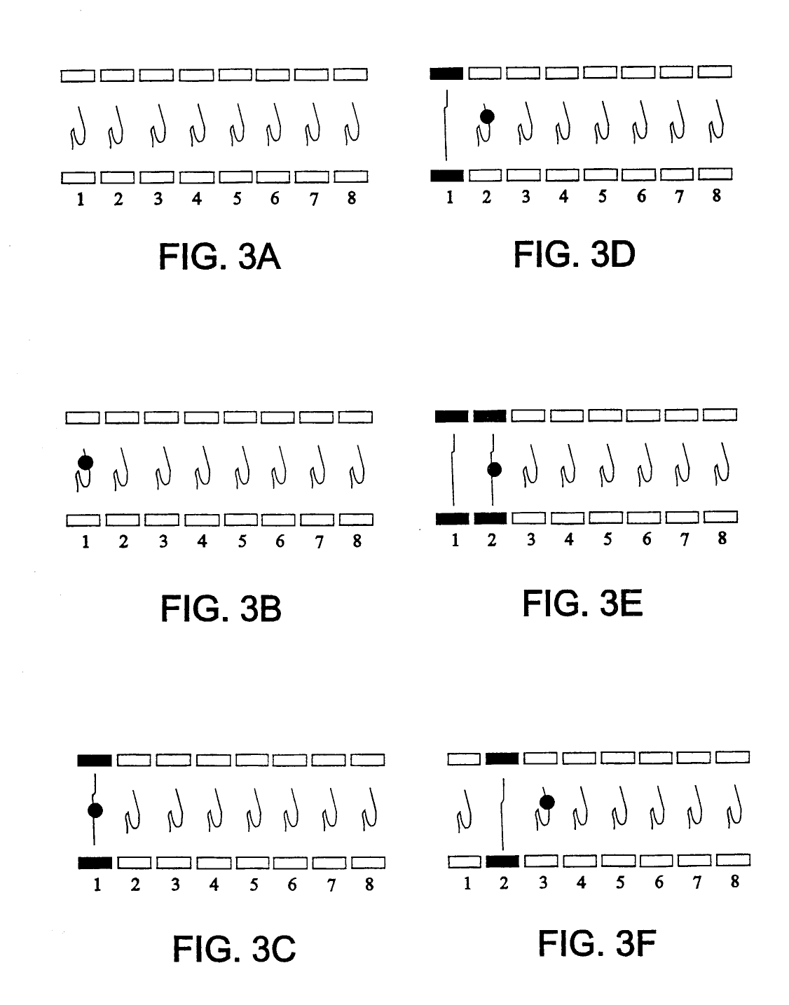

We now give a specific description how a naturally occurring scaffold, namely the FnIII₁₀, can be operated as a FRMR switch. In the case of FnIII₁₀, the G-strand is the first strand to be pulled out while the overall integrity of the remaining FnIII₁₀ module remains essentially unperturbed as illustrated in detail in FIG. 3. This has significant consequences. The RGD loop connecting the G- and F-strand is first shortened at a module's extension of 15±5 Å with respect to its equilibrium state. The loop is then straightened out as the G-strand is pulled away. Shortening of the RGD loop reduces its accessibility to membrane-bound integrins, thus promoting its detachment. Furthermore, straightening of the loop reduces its binding specificity for different members of the integrin family. This change in accessibility and specificity occurs in the early stages of the unfolding pathway while the remaining module maintains a stable or semi-stable configuration. Hence, this molecular device switches the accessibility and binding specificity of its recognition site if a force threshold applied to its C- and N-termini is overcome. The force threshold is dependent on the pulling velocity. The force needs to be sufficiently large to accomplish the shortening and straightening of the RGD loop, but it must not exceed a value which leads to covalent bond breakage within the scaffold's backbone. The scaffold of the FnIII₁₀ or of homologous modules is thus particularly well suited for the rational design of fast regenerable FRMR switches. FRMR switches can, however, also be built utilizing other structural motifs.

Diverse ligands, including but not limited to cell surface molecules (including those in situ), peptides, proteins, polysaccharides, carbohydrates, toxins, polymers, metal ions and metal ion complexes, small molecules, and nucleic acids or oligonucleotides, that recognize FRMR switches can be targeted by functionalizing loops of the FRMR switch with peptide sequences other than the RGD of the specifically exemplified fibronectin domain. For example, the RGD sequence in the loop connecting the B-strands F and G of the FnIII₁₀ module can be replaced by another signaling sequence, ligand binding site, or by an epitope that is specifically recognized by an antibody. The RGD loop can also be replaced by a short sequence that forms a metal binding site, for example. Such a loop can, for example, specifically bind to histidine-tagged proteins. The loop can be designed such that the metal is released upon tension, which will lead to the desorption of the protein. Furthermore, the scaffold can be altered in order to adjust the range of tensions under which the FRMR switch is stretch-regulated. Another highly suited scaffold for the rational design of molecular switches is the anti-receptor antibody fragment (OPG2), which is a member of the Ig family. An advantage of using β-sandwich motifs is that the overall stability of the scaffold enables an accelerated reversible refolding of the FRMR switch after operation.

A variety of approaches allows one to functionalize molecules, materials, or devices with FRMR switches. The FRMR switch is thereby functionalized with reactive groups which are preferentially located at or close to the ends of the module. The FRMR switches release the bound ligands upon stretch-activation. The ligands released upon stretch-activation can be ions, small molecules, peptides, proteins, RNA or DNA, as well as cells and larger particles, among others. Functionalization of materials and devices with FRMR switches can occur by chemical binding of reactive groups on an FRMR switch to the material or device. For example, two reactive sites which are preferentially located at or near the terminal ends of the FRMR switch are bound to two different locations on a viscoelastic object or film that, if deformed or extended, stretches the FRMR switch. Alternatively, one terminus can be attached to a substrate while the other terminus is attached to a bead or another object, including magnetic beads, an optically trapped object, lever arms, or mechanically moveable device surfaces such that the FRMR switch is activated if force is applied to the object. In a further embodiment, one terminus can be attached to a surface or to a molecular assembly while dragging forces pull on the other terminus. Finally, the FRMR switch can also be part of a larger molecule that contains several recognition sites, potentially with recognition sites for different ligands. The FRMRs can be part of a molecule that has been assembled into fibers, networks, membranes, or other materials. Force is transmitted to the FRMR switches as these materials are stretched.

In addition to all the naturally occurring or genetically engineered or chemically synthesized FRMR switches, our invention contemplates integration of naturally occurring FRMR motifs into man-made devices, as well as molecules, containing FRMR switches, added to biological systems for diagnostic purposes.

We have outlined below a few specific examples that illustrate how FRMR switches can be used for practical applications:

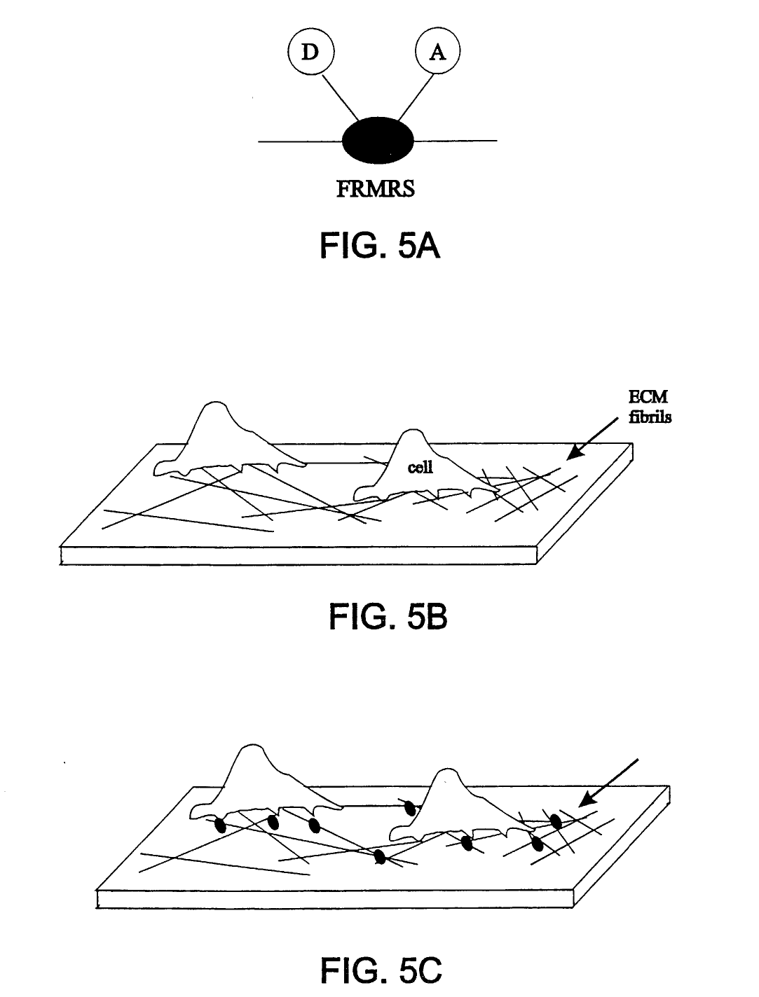

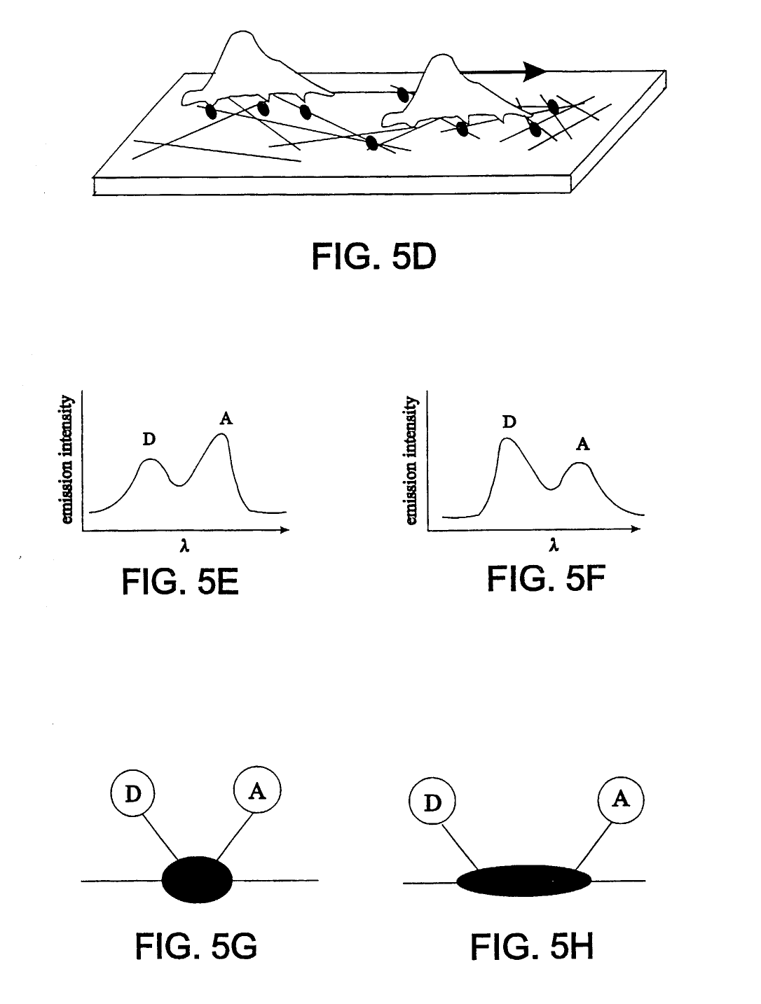

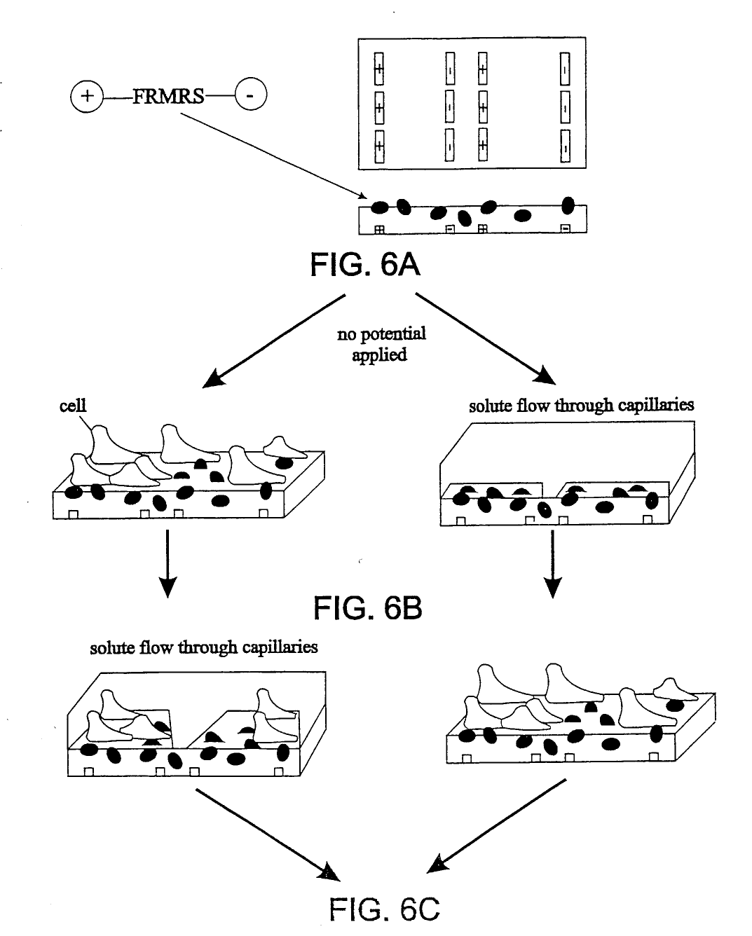

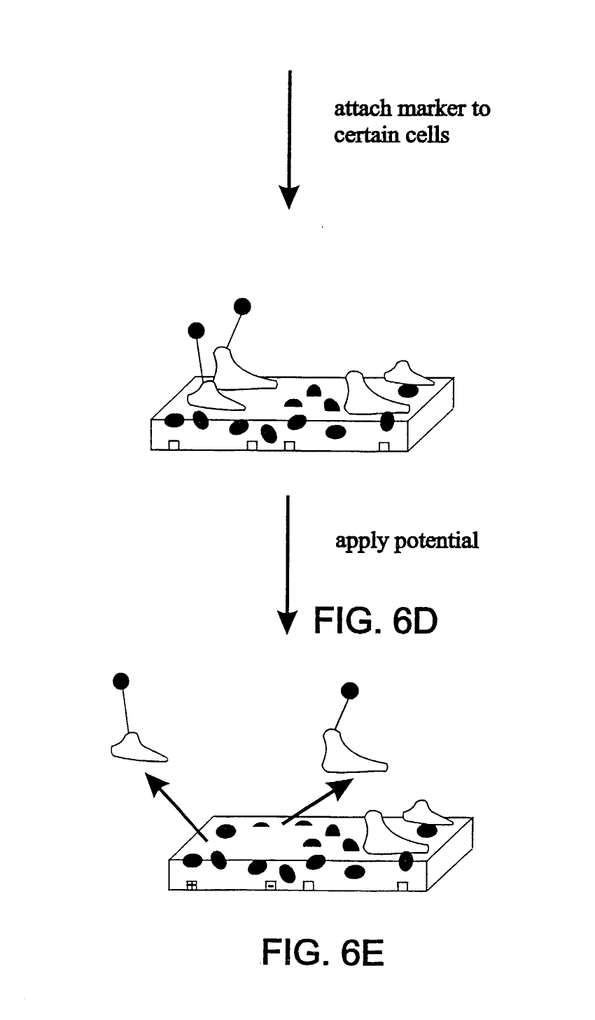

With reference to FIG. 5, this device can be used in various settings. One possibility is that the FRMRS contains the RGD sequence. Cells are then plated on the surface (FIG. 5B-C, left). They are then exposed in a spatially controlled fashion to drugs, pollutants, toxins, cells or other biologically active or ligand molecules. Spatial control of exposure can be accomplished by solute flow through capillaries (FIG. 5C). The cell bed is then exposed to markers that test, for example, for cell survival, cell cycle, gene expression, expression of receptor molecules (FIG. 5D). After inspection of the cell array, cells of interest can be selectively detached from the surface through application of a voltage to the underlying electrodes (FIG. 6E). The potential stretches the FRMRS, thus releasing the cells. Alternatively, the surface of the array can be precoated by drugs, pollutants, toxins or other biologically active molecules in a spatially controlled manner (FIG. 6B-C, right) prior to plating the cells, followed by the procedures as described above.

Referring to FIG. 6, the FRMRS, with acceptor, donor and recognition sites, is incorporated into a thin film of a calorimetric affinity assay. This film is then deposited on top of an array of test molecules (FIG. 6B, side view). The thin film is then ripped off the array. The FRMRs in areas of strong adhesion will be stretch-activated (FIG. 6C). This leads to a locally confined color change (FIG. 6) similar to the color change outlined in FIGS. 5E-5F.

Molecules having FRMR switches can be produced by molecular biological methods using vectors, host cells and cloning, polymerase chain reaction and site-directed oligonucleotide mutagenesis which are well known to the art. Vectors, host cells and reagents are commercially available from sources including, but not limited to, Promega, Madison, Wis.; Stratagene, La Jolla, Calif.; Invitrogen, San Diego, Calif.; Clontech, Palo Alto, Calif.; Pharmacia Biotech, Piscataway, N.J.; among others. Preferred host cells for product of recombinant proteins containing TMR switches include Escherichia coli, Pichya pastoris, Saccharomyces cerevisiae, COS cells, CHO cells, fibroblast cells and others. Alternatively, the switch-containing polypeptides of the present invention can be produced using solid state peptide synthesis with commercially available automated peptide synthesizers (Applied Biosystems, Foster City, Calif., for example) or manual synthesis (e.g., Stewart et al. Solid Phase Peptide Synthesis, Pierce Chemical Company, Rockford, Ill.).

It is understood that the RGD motif of the specifically exemplified TMR switch can be replaced by other binding motifs, especially where a substituted binding motif recognizes a ligand other than that of fibronectin. For example, an epitopic sequence, desirably having 4 to 7 amino acids, can be substituted in place of the RGD motif so that the ligand of the epitopic motif is an antibody with binding specificity for that particular epitope. Another useful substituent is the HIV env-binding region of the human (or simian) CD4 cell surface protein. Such a substituted FRMRS functions in modulated binding and release of HIV or SIV, depending on the CD4 motif used.

Other useful motifs to be placed on the distal end of a loop capable of functioning as a FRMRS include, but are not limited to, calcium or other metal binding sites, a biotin or other vitamin binding site. It is understood that the loop on which the binding site is positioned must be long enough so that the engineered binding site does not interfere with the P-sheet (or β-barrel) secondary structure of the scaffold protein and of a length such that a bound ligand is released in response to “pulling” of the adjacent β-structure or loop.

Where the substituted FRMRS-containing protein is recombinantly produced, it is desirable to modify the wild-type coding sequence so that the region encoding the RGD motif is replaced by a nucleotide sequence encoding the binding motif of interest, for example, by site-directed oligonucleotide mutagenesis or by PCR using a mutagenic primer.

Substituted FRMRS-containing molecules as described above are useful in diagnostic methods and/or in analytical methods and devices. The present FRMRS technology is also applicable to releasable cultured cell growth on a surface coated with FRMRS-containing molecules. Applying tension to the coated surface allows the release of the cultured cells with significantly less mechanical and/or structural damage than conventional release techniques. An example of increased tension would be to cause the swelling of expandable beads coated with TMR-switch containing proteins and specifically bound cells or molecules. Swelling causes increased tension and the release of the bound moieties.

Many of the procedures useful for practicing the present invention, are well known to those skilled in the art of molecular biology. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York, Kaufman (1987) in Genetic Engineering Principles and Methods, J. K. Setlow, ed., Plenum Press, NY, pp. 155-198; Fitchen et al. (1993) Annu. Rev. Microbiol. 47:739-764; Tolstoshev et al. (1993) in Genomic Research in Molecular Medicine and Virology, Academic Press. Abbreviations and nomenclature, where employed, are deemed standard in the field and commonly used in professional journals such as those cited herein.

All references cited herein are hereby incorporated by reference to the extent that they are not inconsistent with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

### Stretch-Activated Scaffolds to be Used in Tissue Engineering

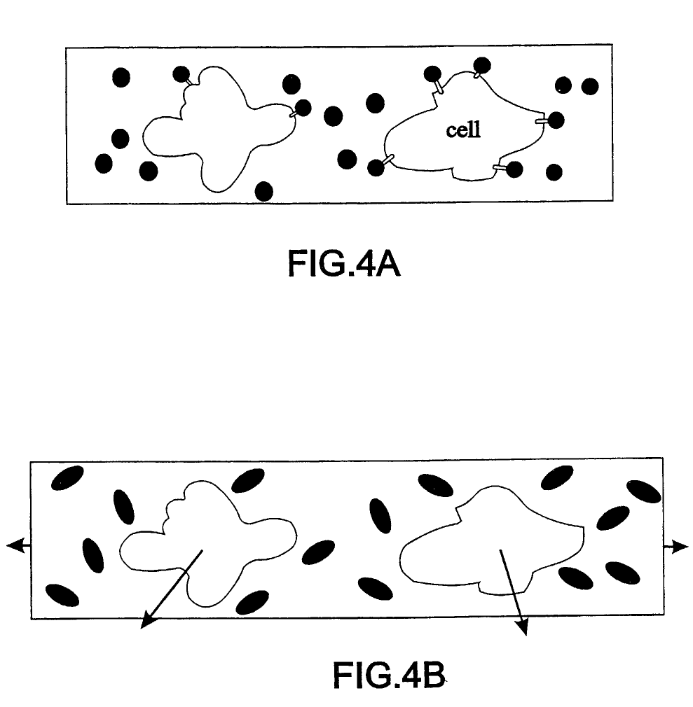

The scaffolds made of biological or synthetic materials contain FRMR switches which expose cell binding domains. A cell-containing scaffold is activated by a stretching motion, which triggers enhanced cell motility (see FIGS. 4A-4B). In the case where the device contains a cell co-culture, it is possible to release only one particular cell type. The FRMR switches are integrated, for example, into the scaffold of an artificial skin. The device is prepared, for example, by allowing cells, potentially of a patient, or cultured cells, to be administered in a therapeutic regimen, to infiltrate the device ex vivo. The cell adhesion strength of the device can be optimized to immobilize the cells, for example, during transport and storage. After stretch activation has occurred the cells start to migrate. Stretch activation can occur, for example, by a surgeon stretching a device just before it is placed into a wound site. The advantage is that the wound closure time is shortened, thereby accelerating the integration of the device into the surrounding skin. Alternatively to being stretched just prior implantation, it is possible to utilize the device such that it is activated only if subject to mechanical strain, for example, after implantation where it replaces blood vessels or other organs. The advantage of using a stretch-activated scaffold is that the density of cell binding sites can be chosen high enough to prevent the cells from migrating out of the device during storage and transport. Cell release and motility can then, however, rapidly be increased at the time of or after implantation without the use of chemical reagents. This is a non-toxic process that does not interfere with the healing process, but rather accelerates healing.

### Example 2

### Process for Cell Sorting

Developing alternate methods for cell sorting is of fundamental interest in biotechnology, biomedical diagnostics and tissue engineering. In most common approaches the cells are separated based on size, shape or mass, either optically or magnetically by utilizing appropriate markers. Our approach using the FRMR switches of the present invention allows separation of cells based on cell adhesiveness. Cells are separated based on a specific surface recognition event which translates into cell adhesion. The FRMR switch contains at least one recognition sequence that is specific to one particular cell type. These FRMR modules are then exposed on a surface of an elastic-deformable device. When a medium containing a mixture of cells flows across the surface of the elastic-deformable device, the targeted cells adhere. For example, one can target melanoma cells by replacing the RGD in the loop in the FnIII₁₀ module by the peptide sequence REDV (SEQ ID NO: 1), or mammary tumor cells by presenting FRGDS (SEQ ID NO:2) in that loop. After the targeted cells have adhered to the surface of the device, stretch activation is used to release them, for example, for further analysis. This novel cell sorting technology is applied to diagnostic methods, sorting cells for use in gene therapy, implantation therapy or to remove harmful (e.g., tumor) cells ex vivo.

### Example 3

### Colorimetric Lit to Rapidly Access Cell Motility for Medical Diagnostics

Cell traction and motility is often altered in malignant cells, for example, in various cancer cells. No fast assays are available that can rapidly probe cell traction and/or motility without major instrumental effort. In this application, cells of interest, for example originating from biopsy or surgery, are cultured in a medium containing tailored molecules which are integrated into the extracellular matrix. The tailored molecules contain one or more donor (D)/acceptor (A) pairs (D-FRMR-A) with a relative distance of not more than 100 Å. A moving cell is capable of stretching its extracellular matrix fibrils as demonstrated experimentally for fibronectin (Ohashi et al. [1999], supra). The spatial distance between A and D increases when external forces induce forced unfolding of the FRMR switch. When D-FRMR-A is integrated into extracellular matrix fibrils, mechanical stretching of the fibrils by cells applies a force on the D-FRMR-A. After excitation of the D with light at its excitation wavelength, the emission spectrum of D is probed. If A has an adsorption spectrum that overlaps with the emission spectrum of D, and if the distance between A and D is less than about 100 Å, it is well known that fluorescence resonance energy transfer occurs from D to A. If the D/A distance is less than about 100 Å within the A-FRMR switch-D in equilibrium, the emission spectrum of this switch is blue-shifted upon stretch-activation, as outlined in FIG. 5F. Typical donor/acceptor (D/A) pairs are commercially available, and include, without limitation, fluorescein/rhodamine and BODIPY/rhodamine (BODIPY is a trademark of Molecular Probes, Inc., Eugene, Oreg. which is a source of D/A pairs useful in the present invention). As an alternative to using dyes as D/A pairs, energy transfer between nanoparticles and dyes, or among nanoparticles, can be employed. Hereby, the size-dependent band gaps of semiconducting nanoparticles, including CdS or the surface plasmon resonances of metal particles, including gold or silver, can be employed. Moving cells are thus distinguished from sessile cells, for example, on the basis of their spectroscopic signature. The fluorescence resonance energy transfer efficiency is thus different for motile cells and sessile cells. This simple fluorescence-based assay utilizes resonance energy transfer processes in order to directly translate cell motility into a color change. It is known that fibronectin, if added to a cell culture medium, is integrated into the extracellular matrix. An example for a tailored molecule is thus wild-type fibronectin or recombinant fibronectin. In this case, donor/acceptor pairs surround those modules that readily unfold when tension is applied, preferentially framing the FnIII₁₀ module. The donor/acceptor groups are chemically bonded to selective sites on or in close proximity to the FRMR switch. Alternatively, fusion proteins can be generated that contain, for example, two different green fluorescence proteins where the emission spectrum of one overlaps with the absorption spectrum of the other.

The procedure, as outlined in FIGS. 5A-5B, involves seeding cells on surfaces. After cell adhesion has occurred, tailored molecules which contain the FRMR switch functionalized with at least one donor/acceptor pair are added to a cell culture medium. Time is allowed for the cells to integrate the tailored molecule into their extracellular matrices, and the emission spectra or ratios at selected wavelengths are monitored while the sample is exposed to light which excites the D. The changes of the emission spectrum can be probed either by integrating the signal from the entire surface, or by detecting it spatially resolved, for example, by the use of a microscope. This is a fast assay to rapidly screen for cell motility, or to visualize those cells out of a large cell colony with an altered speed of migration. It does not require time-lapse video microscopy technology which is currently the most common approach to determine cell motility. This assay is particularly useful to rapidly identify relatively rare target (cancerous, for example) cells within a large cell population.

### Example 4

### Electronically Addressable Array of Biorecognition Sites

FRMR switches are fabricated here on micro- or nanofabricated electrode arrays for use in diagnostics and drug development. It allows controlled release of intact single cells from addressable sites on chip arrays without the use of chemicals or other intruding techniques that may damage the selected cells. These arrays are produced and used in the following manner as outlined in FIGS. 6A-6B.

FRMR switches are functionalized by oppositely charged groups or particles as indicated by ⊕-FRMR-⊖. Each field of the array contains a pair of addressable electrodes such that a potential can be applied to stretch-activate nearby ⊕-FRMR-⊖ switches. These electrode arrays are deposited on a silicon chip, or any surface of choice, e.g., integrated microelectrodes, metaloxide semiconductor field effect transistor (MOSFET) arrays.

The electrode array is covered by a thin film containing ⊕-FRMR-⊖ switches. For example, such a thin film can be a polymer film that contains the ⊕-FRMR-⊖ switches. The ⊕-FRMR-⊖ switches can be incorporated into the polymer film or be located on its surface using a variety of approaches, including covalently cross-linking to the polymer backbone or its side chains, entrapment, and by secondary surface functionalization. Films can include hydrophilic polymers or block copolymers to which proteinaceous molecules can be covalently bound under conditions which do not disrupt secondary and tertiary structure of the FRMRS and which do not deleteriously affect unfolding and refolding of the switch mechanism.

This array can now be used in a variety of different settings, as described below.

First, we describe an array-based testbed where cells are exposed, for example, to a combinatorial mixture of chemicals, including drugs and toxins. For this application, the ⊕-FRMR-⊖ switches contain the RGD sequence and cells are seeded on the surface of the thin film. One way to administer a combinatorial mixture of chemicals is by the use of microfabricated flow channels, for example, within blocks of poly(dimethylsiloxane) (PDMs) (Mrksich et al. [1996] Proc. Natl. Acad. Sci. USA 93:10775-10778). This process of exposing the cells to chemicals via solvent exposure in capillaries can potentially be repeated in a sequential manner by using different chemicals, other capillary geometries, or by different relative positioning of the microchannels on the device surface in subsequent steps. Such microfabrication technology for making microcapillaries is well known to the art. See FIGS. 6A-6B for a diagrammatic representation.

Various methods exist in biotechnology and medicine to interrogate the effect of chemical exposure on cell survival or function, for example by the use of internal or external optical markers for visual cell inspection. After identification of a cell of interest, it can then be selectively lifted off a particular field of the array in a non-intrusive fashion by the application of a voltage to the electrode pair sitting below. The voltage is adjusted such that the ⊕-FRMR-⊖ switches are stretch-activated, thereby detaching a selected cell from the substrate. The cell can now be used for further analysis and/or for cell culturing. This is a simple and cheap technique that can selectively detach individual cells out of a large population of surface cultured cells in a non-intrusive manner without deleteriously affecting viability.

Second, an alternative route of using this basic idea of an array-based testbed where cells are exposed to a combinatorial mixture of chemicals is to first adsorb chemicals to the surface in a combinatorial manner, for example by flow through microcapillaries, or by the generation of various gradients, and then to seed cells onto these pretreated surfaces. The rest of the protocol is as outlined above.

### Example 5

### Colorimetric Array-Based Affinity Assay

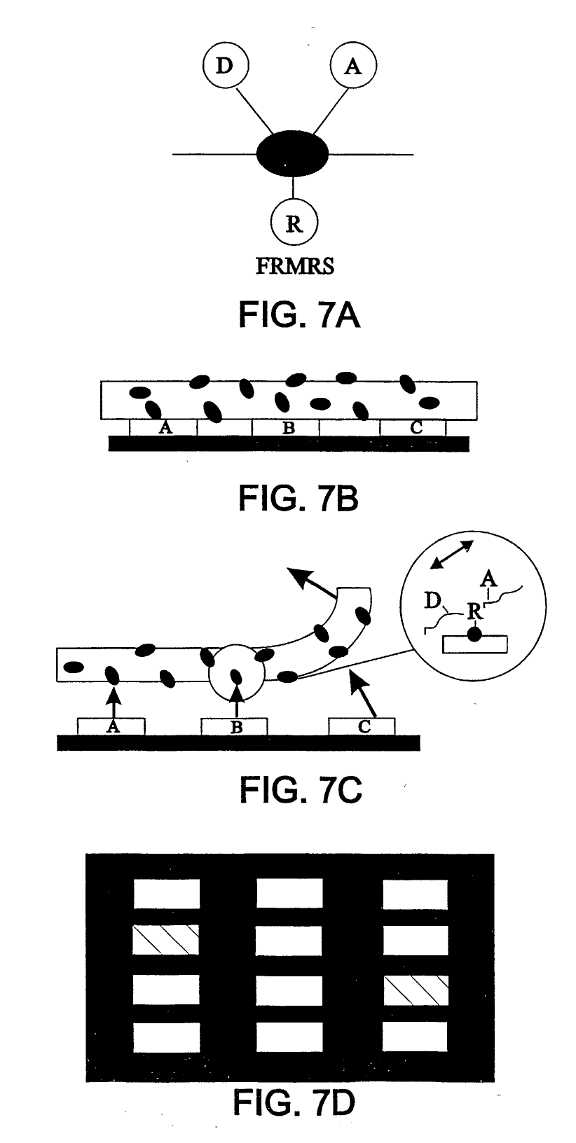

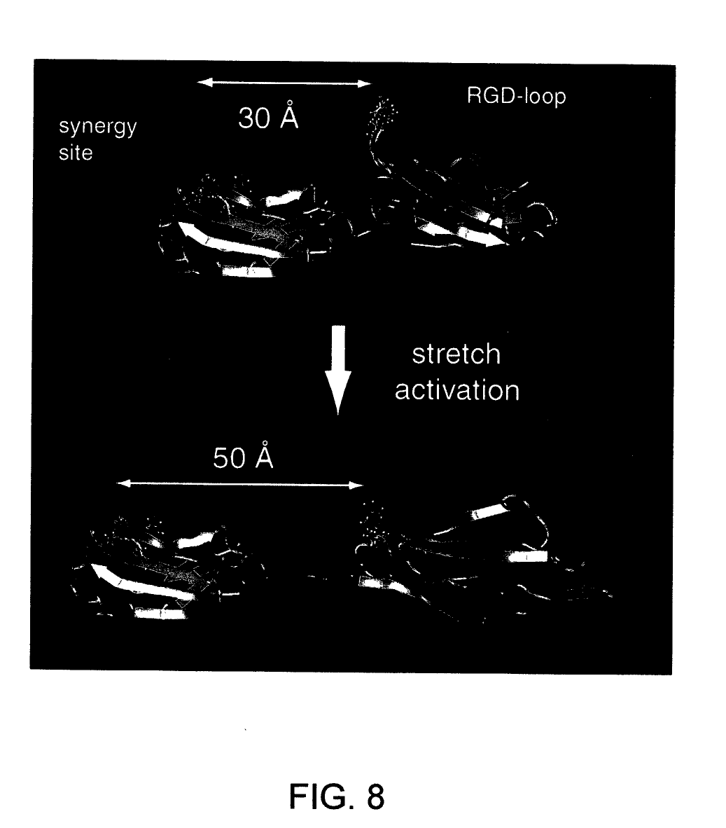

An economical application of the FRMRS technology is a kit as outlined herein that allows a rapid qualitative read-out of binding affinity of test peptides or oligonucleotides arrays where the overall binding strength is translated into a colorimetric response. The array-based testbed contains multiple molecular samples. The test molecules are chemisorbed or physisorbed to the underlying surface of the array. The array is then contacted with a thin matrix that contains D-FRMR-A switches each functionalized with at least one donor/acceptor pair. The D-FRMR-A switches are each covalently bonded to the matrix preferentially by utilizing the two terminal ends of the switch. The matrix can be a transparent polymer film. The thin matrix is then peeled off the array surface. Those points of contact change color where FRMR switches adhere strongly to the test molecules of the array. This results from the fact that the recognition site of the FRMR switches adhere to the array, while the matrix is ripping away its terminal ends. The increased distance between the terminal ends changes the D/A distance, which gives rise to a color change, e.g., a blue shift in the overall emission spectrum. These matrices can be fabricated in forms of tapes functionalized with D-FRMR-A switches. A library of tapes with a variety of signaling sequences spliced into at least one loop of the FRMR switch are fabricated and can be used without requiring access to sophisticated equipment. This embodiment of the present invention is illustrated by FIG. 7.

### Example 6

### Vectorial Molecule-Specific Pump

A vectorial molecule-specific pump can be constructed as a microdevice. A linear array of individually controllable electrodes is constructed, then electrically controllable FRMR switches (switches built with charges on both ends) are anchored in place along the array, as shown in FIG. 3. The electrodes are turned on, then off, moving along the array, thus stretching then releasing FRMR switches. The pump will vectorially move integrins or other ligand molecules that show specific binding to a genetically engineered FRMR switch, and if the integrins are designed as specific carriers, the specific molecules attached to the integrins. The pump makes use of the key FRMR switch qualities of response to local force, molecule specificity, and reversibility.

The vectorial molecule-specific pump can be modified to function for reversible local chemical storage, i.e., as a molecule-specific sponge. Microdevices can be designed to take up and release chemical in a small area, driven by either force or electric signal, for example, where all the FRMRSs switched by the moieties contain bound ligands, and wherein all ligands are simultaneously released as a result of application of voltage across all switches to distort the ligand-binding site or by physically stretching the film, with the same result of releasing the bound ligands. The voltage can be applied by use of a number of small electrodes or one large electrode. The molecule-specific sponge can be adapted to have electronically variable affinity by modulating the electric potential applied across the FRMR switches.

### Example 7

### Biochip to Test Strength of Affinity

A large number of binding affinities can be tested simultaneously by applying forces normal to the surface of a biochip assembled with FRMR switches, for example, fibronectins. Molecule A is attached to surface 1 of a Surface Force Apparatus (SFA) with an FRMR switch, and molecule B is attached to surface 2 by conventional means. Surface 1 is pulled away from surface 2. If A binds strongly to B, a force is exerted on the FRMR switch. Integrins, modified by the attachment of a fluorophor which emits light at a particular known wavelength when the fibronectin or other FRMR switch is stretched, act as a “degree of force experienced” reporter. If A is, instead of one molecule, 900 different molecules placed on a 30×30 array of compartments as in biochips, all 900 binding affinities can be compared with one SFA movement. The compartment n with the best binding affinity between Aₙ and B is the compartment which exerts the most force on the FRMR switch, thus the one which released the most integrin, and thus the compartment which lights up the brightest or otherwise gives the strongest signal.

### Example 8

### Use of Modified Integrins or Integrin Fragments

FRMR switches can be used in a number of areas with the construction of altered integrins or integrin fragments that can bind to the RGD sequence yet also act as carriers for other molecules or as signals to set off molecular cascades. This, for example, includes the coupling of mechanical motion of a microfabricated device to a chemical cascade: motion causes stretch of an FRMR switch, causing unbinding of the integrin, which leads to an increase in integrin concentration, which sets off any chemical cascade one designs. The mechanical motion can also come from electronically controlled stretching, so one can design devices that couple an electrical signal to chemical control. An electrically controlled FRMR switch is constructed by placing oppositely charged groups at both ends of the domain, with mutation or chemical substitution. These switches can then be stretched by turning on and off the local electric field.

In the application described above, integrins can also be integrated into the membranes of membrane vesicles or into the lipid layers of liposomes. The surfaces or the interiors of the liposomes or vesicles can be loaded with signal, triggers or other biologically active molecules.

We claim:

1. A force-regulated molecular recognition switch (FRMRS), said FRMRS comprising a polypeptide having, in linear sequence, a first region of α-helix or β-strand or β-sheet or β-barrel secondary or tertiary structure, an intervening region which acts as a molecular recognition and ligand binding site, and a second region of α-helix or β-strand or β-sheet or β-barrel secondary or tertiary structure, said first and second regions associating with one another such that said intervening region forms a loop and exposes the ligand binding site at the exterior of the polypeptide and such that the association of the first and second regions is reversible and such that a force applied at at least one end of said polypeptide disrupts the association of said first and second α-helix or β-strand or β-sheet or β-barrel secondary structures, wherein binding of a ligand to the ligand binding site can occur when said first and second α-helix or β-strand or β-sheet or β-barrel secondary or tertiary structures are in association with one another to form a stable tertiary structure; said polypeptide being immobilized on a surface of a material or a device such that a force can be applied to said surface to disrupt the secondary or tertiary structure of said force-regulated molecular recognition switch with the result that a ligand bound at the molecular recognition site is released.

2. The FRMRS of claim 1 wherein a polymeric film containing the polypeptide has been deposited onto a surface.

3. The FRMRS of claim 1 wherein said force applied is electrical, mechanical, magnetic or electromagnetic.

4. The FRMRS of claim 1 wherein said force applied is mechanical and results in stretching of the polypeptide and disruption of the association of the first and second regions of α-helix or β-strand or β-sheet or β-barrel secondary or tertiary structures.

5. The FRMRS of claim 1 wherein said ligand binding site comprises the amino acid sequence Arg-Gly-Asp.

6. The FRMRS of claim 1 wherein said ligand binding site comprises the amino acid sequence Arg-Tyr-Asp.

7. The FRMRS of claim 1 wherein the ligand bound is a metal ion or a salt ion.

8. The FRMRS of claim 1 wherein the ligand bound is a peptide, a protein, a cell surface protein, a cell, a polysaccharide, an oligosaccharide, a nucleic acid molecule or a molecule characterized by a molecular weight of less than about 1000 d.

9. The FRMRS of claim 1 wherein said molecular recognition site comprises an epitope recognized by a monoclonal antibody.

10. The FRMRS of claim 1 wherein the ligand is a polyhistidine sequence within a fusion protein.

11. The FRMRS of claim 1 wherein said molecular recognition site comprises the amino acid sequence as given in SEQ ID NO: 1 or SEQ ID NO:2.

12. The FRMRS of claim 1 wherein said molecular recognition site comprises an anti-receptor antibody fragment of OPG2.

13. The FRMRS of claim 1 wherein said polypeptide comprises a multidomain array of recognition sites wherein the spatial distance between at least two of the molecular recognition sites is increased when force is applied to said FRMRS.

14. The FRMRS of claim 1 wherein said FRMRS comprises at least one FnIII domain of fibronectin.

15. A cell motility assay device, said device comprising at least one force regulated molecular recognition switch (FRMRS) of claim 1, wherein said FRMRS comprises at least one integrated energy donor(D)/energy acceptor(A) pair, said FRMRS bound to at least one surface of said device, wherein said FRMRS has a molecular recognition site which functions as a binding site for a ligand associated with a cell or an extracellular matrix of a cell, and such that a force applied to a terminus of said polypeptide disrupts the association of said first and second α-helix or β-strand or β-sheet or β-barrel secondary structures, wherein binding of a ligand to the molecular recognition site can occur when said first and second α-helix or β-strand or β-sheet or β-barrel secondary structures are in association with one another, such that upon optical excitation of D, the emission spectrum is altered after force activation, wherein when said cell moves, force is applied to the FRMRS such that a detectable signal change is generated.

16. The cell motility assay device of claim 15 wherein said ligand is integrin and wherein said FRMRS comprises a binding site which comprises the amino acid sequence Arg-Gly-Asp.

17. The cell motility assay device of claim 15 wherein said FRMRS comprises at least one FnIII domain of fibronectin.

18. A molecule-specific sponge capable of binding a particular target molecule, said sponge comprising a multiplicity of force regulated molecular recognition switches of claim 1, each switch containing a binding site for said target molecule, and further comprising at least one pair of electrodes in contact with one or more force regulated molecular recognition switches such that application of an electrical field across the switches results in release of bound target molecules from the binding sites.

19. The sponge of claim 18 wherein said force regulated molecular recognition switch comprises at least one FnIII domain.

20. An electronically addressable array of force regulated molecular recognition switches (FRMRSs) of claim 1, said array being positioned on a surface within a device, wherein said FRMRSs located within an individual position within said array have a given ligand binding specificity and wherein the positions differ from one another in the ligand binding specificities of the molecular recognition and ligand binding sites.

21. The array of claim 20 wherein each force regulated molecular recognition switch further comprises at least one reporter molecule such that a change in ligand binding status results in a detectable signal change.

22. The array of claim 20 wherein said force regulated molecular recognition switch comprises at least one FnIII domain.

23. The array of claim 20 wherein each FRMRS comprises a ligand binding site and at least one magnetic bead, wherein when said FRMRS is exposed to a magnetic field, the FRMRS experiences a force which stretch-activates the FRMRS with the result that a ligand which was bound to the FRMRS is released.

24. The array of claim 20 wherein each FRMRS can be activated by applying an electric field to said switch.

25. A device for controlled release of selected bound cells, said device comprising a multiplicity of force regulated molecular recognition switches (FRMRS) of claim 1, wherein each FRMRS contains a binding site for a target molecule on a cell surface of a selected cell and is connected to a polymeric network, wherein application of a tensile force onto the polymeric network results in release of the target molecule, resulting in release of the selected bound cell.

26. The device of claim 25 wherein said ligand is integrin and wherein said FRMRS comprises a binding site which comprises the amino acid sequence Arg-Gly-Asp.

27. The device of claim 25 wherein said FRMRS comprises at least one FnIII domain of fibronectin.

28. A device for cell sorting, wherein cell sorting is accomplished by selectively and reversibly binding selected target cells, wherein said device comprises a multiplicity of FRMRSs of claim 1, said FRMRSs having a binding for site for a target ligand on a surface of a selected target cell and wherein said target cell binds to the ligand binding site of the FRMRS, and wherein application of a force to a FRMRS or to a surface of said device, which surface is in contact with a fluid comprising said target cells, results in release of said target cells.

29. The device for cell sorting of claim 28 wherein the ligand binding site comprises the amino acid sequence is as given in SEQ ID NO: 1 when the target ligand is a melanoma cell.

30. The device for cell sorting of claim 28 wherein the ligand binding site is as given in SEQ ID NO:2 when the target ligand is a mammary tumor cell.

31. The device for cell sorting of claim 28 wherein said FRMRSs are activated by application of a force which is mechanical, electric, magnetic or electromagnetic.

32. A device comprising the FRMRS of claim 1 for determining relative binding affinity for ligands and binding partners, wherein said surface is a first surface which is or comprises a thin film comprising a multiplicity of FRMRSs, said device further comprising a second surface on which is immobilized an array of ligands wherein each FRMRS contains a recognition site and an integrated donor/acceptor pair, such that the first surface is first brought into contact with the second surface, resulting in an adhesive contact between the first and second surfaces followed by rapid separation of said surfaces, wherein separation results in a color change of fluorescence emission spectrum of said donor/acceptor pair, whereby areas of high affinity binding between a ligand on the array and the binding partner of the FRMRS are identified.

33. An assay method comprising:
(a) providing at least one force regulated molecular recognition switch (FRMRS) wherein said FRMRS comprises at least one integrated energy donor (D)/energy acceptor (A) pair, said FRMRS bound to at least one surface of said device, wherein said FRMRS has a molecular recognition site which functions as a binding site for a ligand associated with a cell or an extracellular matrix of a cell; and
(b) applying sufficient force to an end of said polypeptide to disrupt the association of said first and second α-helix or β-strand or β-sheet or β-barrel secondary structures and thereby disrupting binding of a ligand to the molecular recognition site.

34. The method of claim 33 also comprising optically exciting said energy donor D such that the optical spectrum is detectably altered after application of said force.

35. The method of claim 33 comprising providing an array of said FRMRSs.

36. The method of claim 33 wherein said applied force is mechanical.

37. The method of claim 33 wherein said applied force is electrical.

38. The method of claim 33 wherein said applied force is electromechanical.

39. The method of claim 33 wherein said applied force is magnetic.

40. The method of claim 33 wherein said FRMRS comprises the amino acid sequence Arg-Gly-Asp.

41. The method of claim 33 wherein said FRMRS comprises the amino acid sequence Arg-Tyr-Asp.

42. The method of claim 33 wherein said FRMRS comprises at least one FnIII domain of fibronectin.

43. A method for determining relative binding affinity for ligands and binding partners, comprising:
(a) providing a device having a first surface comprising the surface of claim 1 having a plurality of FRMRSs immobilized thereon, and further comprising a second surface on which is immobilized an array of ligands, wherein each FRMRS contains a recognition site and an integrated donor-acceptor pair;
(b) contacting said first surface with said second surface, resulting in an adhesive contact between the first and second surfaces;
(c) separating said surfaces, wherein separation results in a color change of fluorescence emission spectrum of said donor/acceptor pair; and
(d) identifying areas of high affinity binding between a ligand on the array and the binding partner of the FRMRSs.
